(19) United States
(12) Patent Application Publication
(10) Pub. No.: US 2018/0218309 A1
(43) Pub. Date: Aug. 2, 2018

(54) PERSONALITY-BASED COGNITIVE TEAM MEMBER SELECTION

(71) Applicant: INTERNATIONAL BUSINESS MACHINES CORPORATION, Armonk, NY (US)

(72) Inventors: Martin G. Keen, Cary, NC (US); Adam J. Smye-Rumsby, Reading, PA (US)

(21) Appl. No.: 15/422,900

(22) Filed: Feb. 2, 2017

(51) Int. Cl. G06Q 10/06; A61B 5/16

(52) U.S. Cl. CPC G06Q 10/063118; A61B 5/167

(57) ABSTRACT

Systems and methods for personality-based cognitive team member selection are disclosed. In embodiments, a computer-implemented method, comprises: retrieving from multiple remote data resources, by a computing device, communication data authored by a participant; processing, by the computing device, the communication data to produce formatted data; analyzing, by the computing device, the formatted data to obtain personality trait scores for corresponding personality traits based on the formatted data; generating, by the computing device, a user profile for the participant including the personality trait scores; and storing, by the computing device, the user profile in a database.

### BACKGROUND

The present invention relates generally to team member selection and, more particularly, to personality-based cognitive team member selection.

When forming new teams in a business environment, it can take time and effort to identify the best team composition. Existing methods of forming teams place an emphasis on selecting the required skills and experience, and focus less on ensuring team members complement each other on more fundamental levels, such as personality. Assembling an ineffective team increases the risk that the team's goals and objectives will not be achieved.

Teams are a common construct for completing complex or time-consuming tasks that are impractical for an individual to attempt alone. The differing and complementary skills of a group of individuals are combined in a team. Teams are often assembled for specific, time-boxed initiatives such as a project to design a new piece of computer software.

Some hiring entities conduct psychometric testing of candidates in order to identify those whose personalities may not align sufficiently with the desired character traits of the ideal candidate. A variety of such tests have been devised, with many taking the form of a structured questionnaire that is completed by a candidate in a supervised setting.

### SUMMARY

In an aspect of the invention, a computer-implemented method includes: retrieving from multiple remote data resources, by a computing device, communication data authored by a participant; processing, by the computing device, the communication data to produce formatted data; analyzing, by the computing device, the formatted data to obtain personality trait scores for corresponding personality traits based on the formatted data; generating, by the computing device, a user profile for the participant including the personality trait scores; and storing, by the computing device, the user profile in a database.

In another aspect of the invention, there is a computer program product for personality-based cognitive team member selection. The computer program product comprises a computer readable storage medium having program instructions embodied therewith. The program instructions are executable by a computing device to cause the computing device to: obtain a user profile for a participant, the user profile including personality trait scores for corresponding personality traits; obtain user profiles for respective team members, each of the user profiles including personality trait scores for the corresponding personality traits; compare the participant's personality trait scores with the personality trait scores of each team member; determine that a difference between at least one of the personality traits of the participant and one of the corresponding personality traits of at least one respective team member meets a predetermined threshold value based on the comparing; and record the difference in an event log as an event.

In another aspect of the invention, there is a system for personality-based cognitive team member selection. The system includes a CPU, a computer readable memory and a computer readable storage medium associated with a computing device. The system further concludes program instructions to obtain a user profile for a participant, the user profile including personality trait scores for corresponding personality traits; program instructions to obtain user profiles for respective team members, each of the user profiles including personality trait scores for the corresponding personality traits; program instructions to calculate mean personality trait scores for each of the plurality of personality traits across all team members to obtain a first set of team scores; program instructions to calculate personality trait scores for each of the plurality of personality traits across all team members plus the participant to obtain a second set of team scores; program instructions to compare the first set of team scores to the second set of team scores; program instructions to determine that a difference between personality traits of the first set of team scores and corresponding personality traits of the second set of team scores meets a predetermined threshold value based on the comparing; and program instructions to record the difference in an event log as an event; wherein the program instructions are stored on the computer readable storage medium for execution by the CPU via the computer readable memory.

### BRIEF DESCRIPTION OF THE DRAWINGS

The present invention is described in the detailed description which follows, in reference to the noted plurality of drawings by way of non-limiting examples of exemplary embodiments of the present invention.

FIG. 1 depicts a computing infrastructure according to an embodiment of the present invention.

FIG. 2 shows an exemplary environment in accordance with aspects of the invention.

FIG. 3 shows a flowchart of steps of a method in accordance with aspects of the invention.

FIG. 4 shows an exemplary user profile in accordance with aspects of the invention.

FIGS. 5A and 5B show a flowchart of steps of a method in accordance with aspects of the invention.

FIG. 6 depicts an exemplary recommendation sent from the recommendation module.

### DETAILED DESCRIPTION

The present invention relates generally to team member selection and, more particularly, to personality-based cognitive team member selection. Embodiments of the present invention recognize that forming effective teams can be a time-consuming and error-prone task. Team leaders or supervisors often have to spend time interviewing several potential team members for a particular position when selecting the individuals that will comprise a new team. The interview setting does not necessarily provide an accurate representation of how a candidate will perform under the pressures of a project schedule complete with milestones and deadlines. Nor does it allow a team leader to assess the quality of a candidate's interaction with potential team mates. Poor decisions when forming teams can lead to issues such as personality conflicts between team members, causing disruptions and inefficiencies that could jeopardize a team's ability to meet its objectives. Embodiments of the present invention also recognize that the testing of potential team members or employees can be costly to administer, often requiring the oversight of trained staff. It can also be subject to bias and error, since a candidate is usually aware they are under close scrutiny and may provide answers that they feel improve their chances of being selected, rather than answers that accurately reflect their true opinion. Such methods also assume the organization has taken the time to create an accurate personality profile of the ideal candidate to use as a benchmark. In practice, this may not always be the case.

In embodiments, systems and methods of the present invention apply insight into team dynamics to predict potential areas of conflict due to factors such as differences in personality, based on analysis of everyday communications. Embodiments of the present invention enable leaders to make better team selection decisions that support increased team effectiveness, or even allow for team selection to be automated, saving additional time over current methods of composing teams.

Advances in technology now permit accurate machine determination of personality based on analysis of an individual's amassed body of everyday communications and writings, such as emails, instant messages, Short Message Service (SMS) messages, blog or social media posts, etc. The communications may encompass communications authored on a range of different dates and times. In most cases such communications were not written with the specific objective of impressing a recruiter and so personality assessment derived from analysis of these communications could be considered less subject to bias compared to psychometric tests. Indeed, some communications could have been authored several years or even decades prior to being collected for purposes of assessment of personality. By analyzing the personality of a candidate and comparing it to the rest of a team during a pre-hire or team-building phase, a system of the present invention can notify team leaders, recruiters and other stakeholders of differences in personality that could be predictors of increased potential for personality conflicts.

In embodiments, the present invention provides a technical solution to the problem of building teams. In aspects, a system is provided that retrieves participant-originating digital data and analyzes the data to determine the desirability of combining various participants in a team environment.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

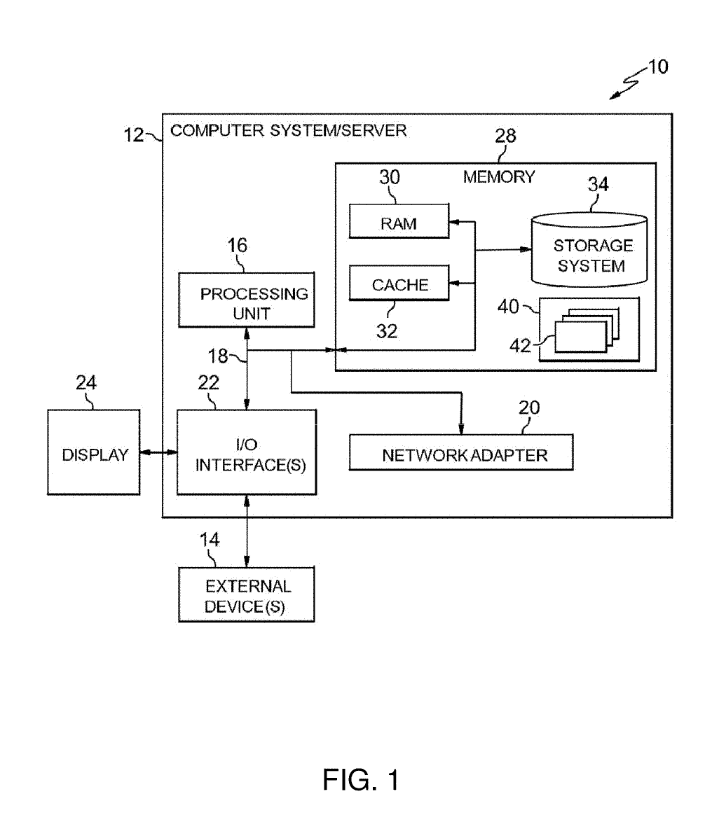

Referring now to FIG. 1, a schematic of an example of a computing infrastructure is shown. Computing infrastructure 10 is only one example of a suitable computing infrastructure and is not intended to suggest any limitation as to the scope of use or functionality of embodiments of the invention described herein. Regardless, computing infrastructure 10 is capable of being implemented and/or performing any of the functionality set forth hereinabove.

In computing infrastructure 10 there is a computer system (or server) 12, which is operational with numerous other general purpose or special purpose computing system environments or configurations. Examples of well-known computing systems, environments, and/or configurations that may be suitable for use with computer system 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 12 may be described in the general context of computer system executable instructions, such as program modules, being executed by a computer system. Generally, program modules may include routines, programs, objects, components, logic, data structures, and so on that perform particular tasks or implement particular abstract data types. Computer system 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system 12 in computing infrastructure 10 is shown in the form of a general-purpose computing device. The components of computer system 12 may include, but are not limited to, one or more processors or processing units (e.g., CPU) 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system 12 typically includes a variety of computer system readable media. Such media may be any available media that is accessible by computer system 12, and it includes both volatile and non-volatile media, removable and non-removable media.

System memory 28 can include computer system readable media in the form of volatile memory, such as random access memory (RAM) 30 and/or cache memory 32. Computer system 12 may further include other removable/non-removable, volatile/non-volatile computer system storage media. By way of example only, storage system 34 can be provided for reading from and writing to a nonremovable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system 12 may also communicate with one or more external devices 14 such as a keyboard, a pointing device, a display 24, etc.; one or more devices that enable a user to interact with computer system 12; and/or any devices (e.g., network card, modem, etc.) that enable computer system 12 to communicate with one or more other computing devices. Such communication can occur via Input/Output (I/O) interfaces 22. Still yet, computer system 12 can communicate with one or more networks such as a local area network (LAN), a general wide area network (WAN), and/or a public network (e.g., the Internet) via network adapter 20. As depicted, network adapter 20 communicates with the other components of computer system 12 via bus 18. It should be understood that although not shown, other hardware and/or software components could be used in conjunction with computer system 12. Examples, include, but are not limited to: microcode, device drivers, redundant processing units, external disk drive arrays, RAID systems, tape drives, and data archival storage systems, etc.

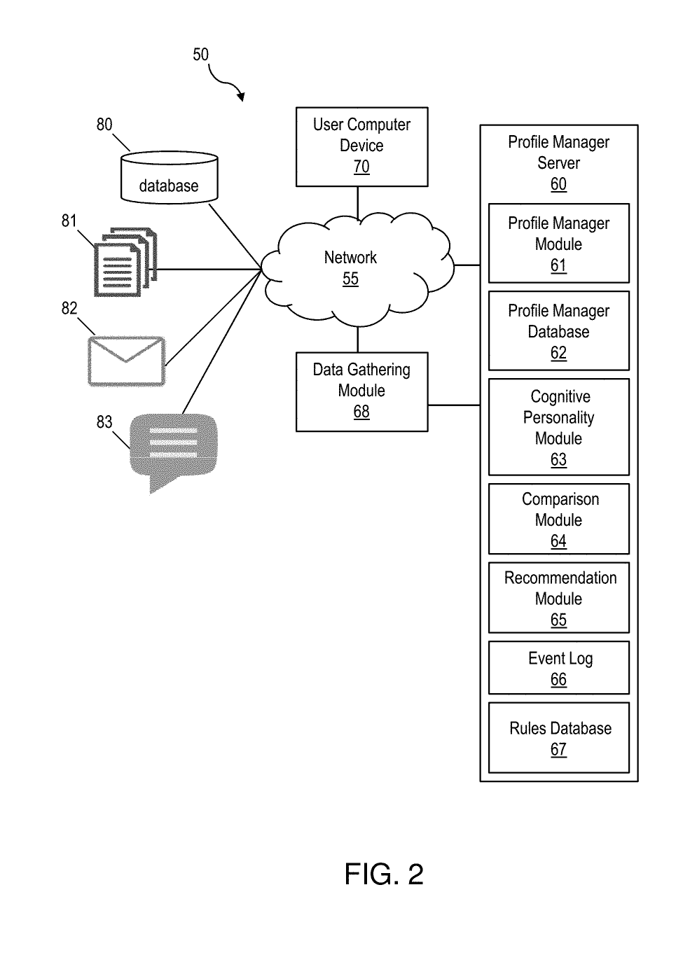

FIG. 2 shows an exemplary system 50 for personality-based cognitive team member selection in accordance with aspects of the invention. The system 50 is shown in an exemplary environment including a profile manager server 60 connected to a network 55. The profile manager server 60 may comprise a computer system 12 of FIG. 1, and may be connected to the network 55 via the network adapter 20 of FIG. 1. The profile manager server 60 may be configured as a special purpose computing device. For example, the profile manager server 60 may be configured to receive data from a data gathering module 68, either directly, or through the network 55. The data gathering module 68 may comprise components of the computer system 12 of FIG. 1, or may be part of the profile manager server 60. In embodiments, the data gathering module 68 is a cloud-based module managed by a third party and configured to provide data to the profile manager server 60 through the network 55. A user computer device 70 may be in communication with the profile manager server 60 and/or the data gathering module 68.

The network 55 may be any suitable communication network or combination of networks, such as a local area network (LAN), a general wide area network (WAN), and/or a public network (e.g., the Internet). The user computer device 70 may be a general purpose computing device, such as a desktop computer, laptop computer, tablet computer, smartphone, etc. In embodiments, the profile manager server 60 is adapted to send a team member selection report to the user computer device 70.

Still referring to FIG. 2, data gathering module 68 is in communication with a plurality of data resources represented at 80-83. In embodiments, data resources 80-83 are remote resources accessed through the network 55. Exemplary data resources are shown to include a database or database server 80 (e.g., human resources database), a social network application or server 81, an email application or server 82, and an instant messaging application or server 83; however, the data resources utilized by the invention may be any resource that provides communication data authored by a participant. Such communication data may include emails, social network postings, resumes, cover letters, instant messages, etc. In embodiments, the data gathering module 68 is adapted to retrieve communication data authored by a participant from the user computer device 70. In embodiments, the data gathering module 68 is adapted to process the communication data received from one or more data resources (e.g., 80, 81, 82, 83) into an appropriate format, and prepares the data for analysis by the profile manager server 60.

Referring to FIG. 2, a profile manager module 61 of the profile manager server 60 is configured to perform one or more of the functions described herein. The profile manager module 61 may include one or more program modules (e.g., program module 42 of FIG. 1) executed by the profile manager server 60. In embodiments, the profile manager module 61 is configured to receive formatted communication data authored by a participant from the data gathering module 68, and generate a user profile for the participant. In aspects, the profile manager module 61 feeds formatted communication data to a cognitive personality module 63 of the profile manager server 60. The cognitive personality module 63 may include one or more program modules (e.g., program module 42 of FIG. 1) executed by the profile manager server 60. In aspects, the cognitive personality module 63 is configured to analyze the formatted communication data to determine personality trait scores for the participant, and feed the personality trait scores to the profile manager module 61 to be incorporated into the participant's user profile. In aspects, the user profiles generated by the profile manager module 61 are stored in a profile manager database 62.

Referring to FIG. 2, a comparison module 64 of the profile manager server 60 is configured to perform one or more of the functions described herein. The comparison module 64 may include one or more program modules (e.g., program module 42 of FIG. 1) executed by the profile manager server 60. In embodiments, the comparison module 64 is configured to retrieve user profiles from the profile manager database 62 and compare personality trait scores from the user profiles. The comparison module 64 recognizes certain data events indicating a potential conflict, and stores the data events in an event log 66. In embodiments, a recommendation module 65 may include one or more program modules (e.g., program module 42 of FIG. 1) executed by the profile manager server 60. In aspects, the recommendation module 65 utilizes rules from a rules database 67 to determine the nature of any potential conflicts indicated by the stored event data in the event log 66. In embodiments, the recommendation module 65 generates a recommendation report, and sends the report to an appropriate party.

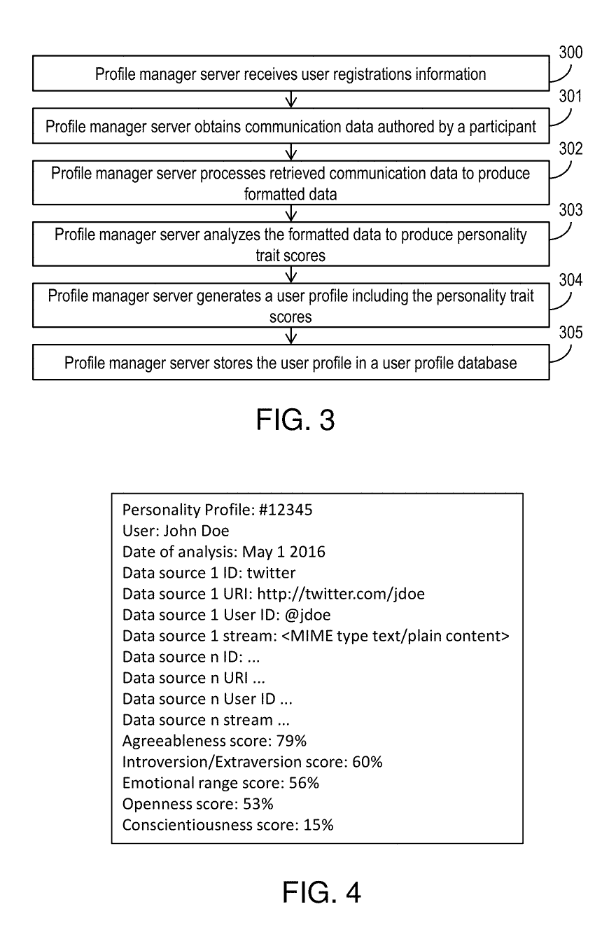

FIG. 3 shows a flowchart of a method in accordance with aspects of the invention. Steps of the method of FIG. 3 may be performed in the environment illustrated in FIG. 2, and are described with reference to elements shown in FIG. 2.

At step 300, the profile manager server 60 receives user registration information. In embodiments, an administrator enters user registration information into the profile manager server 60. Alternatively, the profile manager server 60 can retrieve user registration information from the data gathering module 68. User registration information may include a participant's identifying information (e.g., name, birth date, etc.), permissions to use passwords, and identification of passwords (e.g., email and social media passwords).

At step 301, the profile manager server 60 obtains communication data authored by the registered participant. In embodiments, the data gathering module 68 obtains written communication data from one or more data resources 80-83. Written communication data can be any data authored by a participant, including written data such as resumes, cover letters, email communications, instant messages, blog posts, social media posts, etc. In embodiments, the profile manager server 60 obtains communication data authored over a range of dates and times. For example, the data gathering module 68 may obtain emails, blog posts and social media posts written over a period of two years, such that accumulated writings of a participant may be analyzed in accordance with embodiments of the invention. Data gathering module 68 may utilize user registration information stored in the profile manager server 60, to access the communication data from the resources 80-83. By way of example, the profile manager server 60 may utilize permissions to access email and social media accounts along with passwords gathered at step 300 to access the participant's email correspondence and social media postings. In embodiments, no permissions are necessary, and the profile manager server 60 utilizes only publicly available communication data (digital data).

At step 302, the data gathering module 68 of the profile manager server 60 processes the retrieved communication data to produce formatted data. In aspects, the processing comprises the data gathering module 68 normalizing communication data retrieved at step 301.

At step 303, the profile manager server 60 analyzes formatted data of the participant to produce personality trait scores for the participant. In embodiments, the personality trait scores include scores for the so-called Big Five Factors, including: openness, conscientiousness, extroversion, agreeableness and neuroticism. In embodiments, the personality trait scores include scores for subcategories of the Big Five Factors. By way of example, the personality trait of agreeableness may be further parsed into the personality traits of: altruism, cooperation, modesty, compromise, sympathy and trust. In aspects, the cognitive personality module 63 analyzes formatted data from step 302 to determine the author's (participant's) personality trait scores using an available text analyzing program. Although shown as part of the profile manager server 60, the cognitive personality module 63 may be a remote module provided by a third party, for example.

At step 304, the profile manager module 61 of the profile manager server 60 generates a user profile for the participant, including the personality trait scores from step 303 and user registration information from step 300.

At step 305, the profile manager server 60 stores the user profile generated at step 304 in a database. In aspects, the profile manager module 61 stores user profiles in the profile manager database 62. The profile manager server 60 may utilize user profiles stored in the profile manager database 62 to provide a baseline for comparison with other profiles. In aspects, the profile manager server 60 may update user profiles periodically to track how a participant's personality develops over time (e.g., how Big Five Factors change over time).

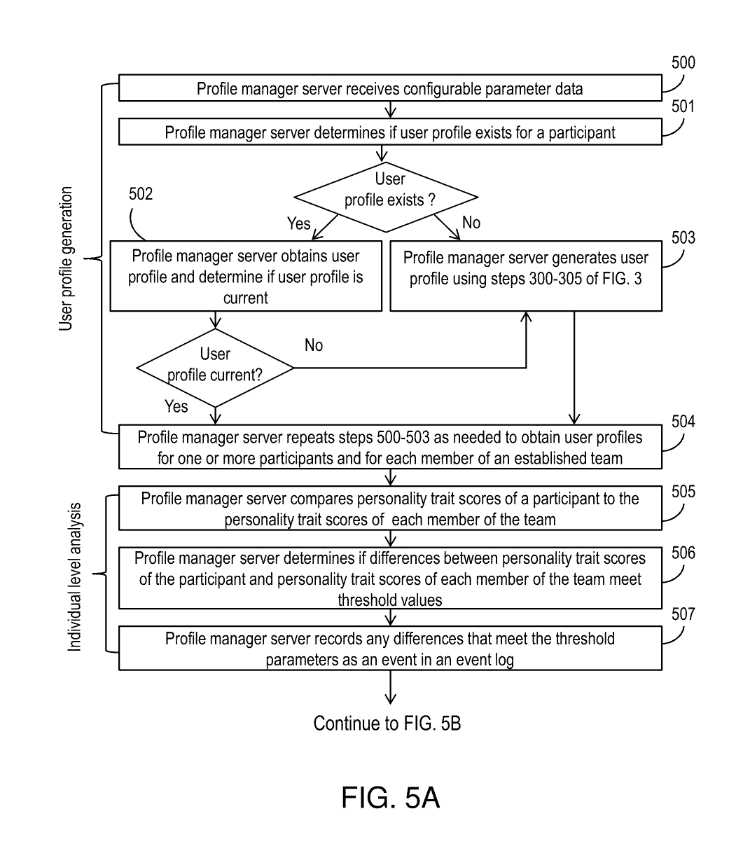

FIG. 4 depicts an exemplary user profile in accordance with embodiments of the invention. In the example shown, a user profile for the participant John Doe includes a personality profile number, date the user profile was generated, data resources from which communication data was obtained, user identification information for each data resource, and personality trait scores for the factors of agreeableness, introversion/extroversion, emotional range, openness and conscientiousness.

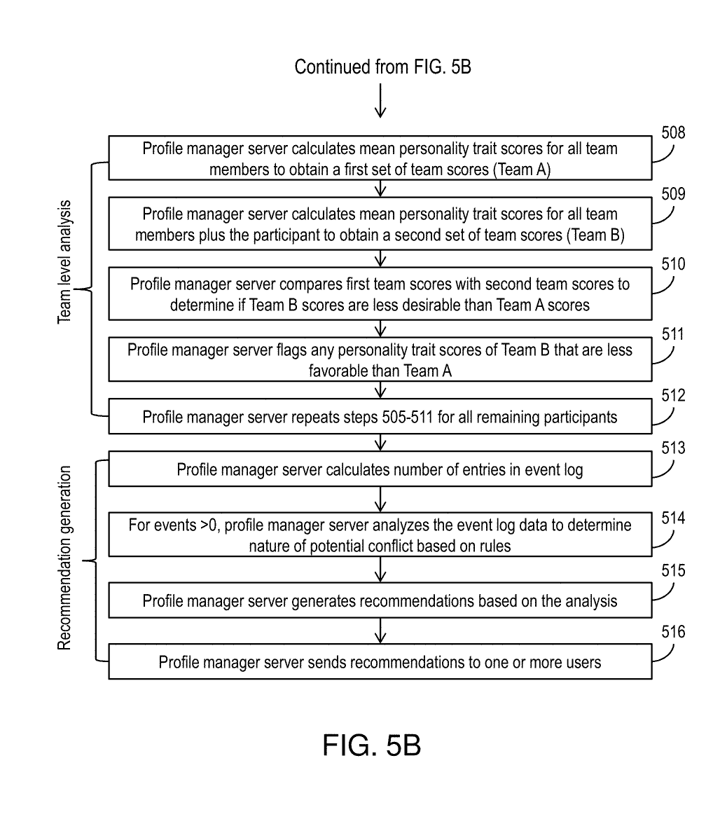

FIGS. 5A-5B show a flowchart of a method in accordance with aspects of the invention. More specifically, FIGS. 5A-5B illustrate a method for obtaining team member selection recommendations. Steps of the method of FIGS. 5A and 5B may be performed in the environment illustrated in FIG. 2, and are described with reference to elements shown in FIG. 2.

Two modes of operation are illustrated by FIGS. 5A and 5B. In a first mode, a team already exists in reality. In this first mode, personality profiles are likely already available for each team member through pre-hiring or on-boarding processes. In a second mode, a team member selection analysis is to be conducted against hypothetical teams. The membership of such a team can be either fully or partially specified by an administrator using manual means, or in an automated fashion using the recommendation module 65 (discussed in more detail below). Thus, a team does not necessarily exist outside of the system 50 and user profiles may not be available for each hypothetical team member. In this latter scenario, the profile manager server 60 can generate user profiles for each hypothetical team member in real-time as required by the profile manager module 61, and then stored for future retrieval in the profile manager database 62.

At step 500, the profile manager server 60 receives configurable parameter data for the system 50. Configurable parameter data includes any parameter of the system 50 that may be adjusted to obtain optimal team member recommendations. In aspects, an administrator can enter configurable parameter data into the profile manager server 60. In embodiments, the configurable parameter data includes threshold values (e.g., acceptable ranges) for personality traits (e.g., Big Five Factors) and user profile expirations dates (indicating when a user profile should be considered old or expired).

At step 501, the profile manager server 60 determines if a user profile exists for a participant. In embodiments, the profile manager server 60 determines if a user profile for the participant is stored in the profile manager database 62.

At step 502, if the profile manager server 60 determines that a user profile exists, the profile manager server 60 obtains and analyzes the user profile to determine if the user profile is current. In embodiments, the profile manager module 61 determines if profile generation data in the user profile meets an expiration threshold value (configurable parameter data) received at step 500. For example, an administrator may set the threshold value at one year after the profile generation data, such that the profile manager server 60 would consider any user profiles generated more than a year earlier to be expired.

At step 503, if the profile manager server 60 determines that a user profile does not exist at step 501, or if the profile manager server 60 determines that the user profile is not current at step 502, then the profile manager server 60 generates a user profile in accordance with steps 300-305 of FIG. 3.

At step 504, the profile manager server repeats steps 500-503 as needed to obtain user profiles for one or more participants and for each member of an established team. The term established team refers to a team established in advance by an administrator (hypothetical or real) or a hypothetical team generated as a recommended team by the recommendation module 65.

At step 505, the profile manager server 60 compares the personality trait scores of a participant to the personality trait scores of each member of the team. By way of example, the comparison module 64 may compare a participant's agreeableness score, introversion/extroversion score, emotional range score, openness score and conscientiousness score with corresponding scores of each team member. In embodiments, for each personality trait, the comparison module 64 generates a graph representative of a distance between the personality trait score of the participant and the personality trait score of the team member.

At step 506, the profile manager server 60 evaluates differences between personality trait scores generated at step 505 to determine if they meet a threshold value. In embodiments, the comparison module 64 iterates through personality trait scores, comparing each team member's scores to the participant's scores. In embodiments, the comparison module 64 evaluates the difference between each personality trait of the participant and the team member to determine if the different meets a respective threshold value (configurable parameter data) received at step 500. In embodiment, the comparison module 64 calculates the Euclidean distance between team member and participant scores. By way of example, an administrator may establish a threshold value for agreeableness such that if a difference between the agreeableness of the participant and the team member is greater than 10%, it meets the threshold value.

At step 507, if the profile manager server 60 determines at step 506 that the distance is outside a predetermined acceptable range or threshold value, the comparison module 64 records the difference as an event in the event log 66. In aspects, each event in the event log 66 represents a potential personality conflict. In embodiments, event data recorded in the event log 66 by the comparison module 64 includes participant identification, team member identification, personality trait name, the distance and the acceptable threshold value or range, for use by the recommendation engine 65 at a later stage.

With reference to FIG. 5B at step 508, the profile manager server 60 calculates mean (i.e., average) personality trait scores for all team members (hypothetical or real) to obtain a first set of team scores (Team A). In embodiments, the comparison module 64 calculates mean Big Five Factor scores for all team members.

At step 509, the profile manger server 60 calculates mean personality trait scores for all team members (hypothetical or real), plus the participant, to obtain a second set of team scores (Team B). In embodiments, the comparison module 64 calculates mean personality trait scores using user profiles stored in the profile manager database 62. This step results in two sets of team-level Big Five Factor scores; the first set for the team not including the participant (Team A), and the second set representing the team with the participant as a member of the team (Team B).

At step 510, the profile manager server 60 compares the first team scores (Team A scores) with the second team scores (Team B scores) to determine if any personality trait scores of Team B are less favorable than Team A. In an ideal scenario, including the participant in the team improves the team-level personality.

At step 511, the comparison module 64 of the profile manager server 60 flags any personality trait where the score from the second set is (Team B) determined in step 511 to be less favorable than the equivalent in the first set (Team A), and records the data in the event log 66 as an event. In aspects, the event data includes identification of the participant, identification of the team members in the first set (Team A), the personality trait flagged, the distance, and the acceptable threshold value or range, for use by the recommendation engine 65 in a later stage.

At step 512, the profile manager server 60 repeats steps 505-511 for all remaining participants. It can be understood that multiple team member scenarios could be analyzed with the method of FIGS. 5A and 5B to provide a user with recommendations regarding a number of possible teams. Thus, embodiments of the present invention provide automated identification and analysis of personality differences, as well as predictive analysis of personality traits to predict a degree of complementary fit of a subject within a group and to predict the success of a particular team given the members of the team. It should be understood that embodiments of the present invention enable the creation of composite, group, or team-level personality profiles (steps 505-507 or steps 508-512) from a collection of individual profiles (steps 500-504).

At step 513, the profile manager server 60 calculates the number of entries (events) in the event log 66. In embodiments, the recommendation module 65 analyzes the entries in the event log 66 to determine if the number of entries is greater than zero. If the number of entries in the event log 66 is zero, the implication is that there is no specific area of personality that suggests a higher than average risk of future personality conflict within any of the teams evaluated.

At step 514, if the recommendation module 65 of the profile manager server 60 determines that the number of entries (events) in the event log 66 is greater than zero, the recommendation module 65 analyzes the event log data to determine the nature of potential conflicts. In embodiments, the recommendation module 65 applies rules to guide its output. In aspects, the recommendation module 65 utilizes rules from the rules database 67, wherein the rules are configurable parameters received by the profile manager server 60 at step 500. By way of example, a user may enter a rule in the rules database 67 that states: if the count of individual member-level analysis events (steps 505-507) is non-zero and the count of team-level analysis events (steps 508-511) is also non-zero, then there is an elevated risk that: (1) the participant's personality could conflict with one or more team members; and (2) the participant's personality would negatively impact the team's collective personality.

At step 515, the recommendation module 65 of the profile manager server 60 generates recommendations based on the analysis of step 514. In an exemplary scenario where the recommendation module 65 determines that the count of individual member-level analysis events is non-zero, and the count of the team-level analysis events is non-zero, the recommendation engine 65 would output guidance that adding the participant to the team would not be recommended in the interest of team cohesion and harmony.

At step 516, the profile manger server 60 sends recommendations to one or more users. In embodiments, the recommendation module 65 sends the recommendation as a push notification, email, text message, or the like. By way of example, the recommendation module 65 may send a team leader a push notification informing them that a participant (e.g., potential employee) is not a good fit for a team. In embodiments, the recommendation module 65 sends the recommendation to the user computer device 70. It should be understood that embodiments of the present invention provide for automated analysis of both individual level (steps 504-507) and group-level (508-512) personalities to generate insights into a degree of complementary fit of an individual subject within a group in accordance with steps 513-516.

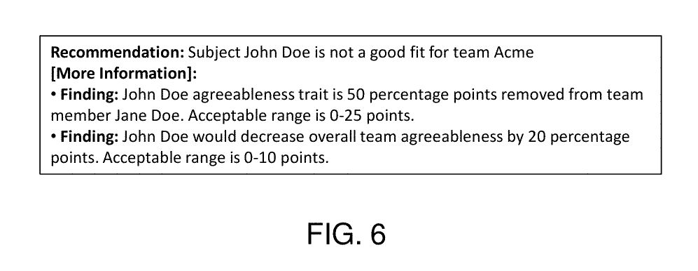

FIG. 6 depicts an exemplary recommendation sent from the recommendation module 65. In the example shown, a user receives a recommendation from the profile manager server 60 that a potential team member John Doe is not a good fit for the established team Acme. The example shown provides information regarding the individual-level recommendations, including the fact that: John Doe's agreeableness trait is 50 percentage points removed from Jane Doe (a member of team Acme), and the acceptable range is 0-25 points. The example shown also provides team-level recommendations, including the fact that: John Doe would decrease the overall team agreeableness by 20 percentage points, and the acceptable range is 0-10 points. Thus, embodiments of the invention provide automated creation of insight and suggested actions to a user.

Advantageously, embodiments of the invention enable: (1) application of available automated analysis of a candidate's amassed body of everyday communications and writings, such as emails, instant messages, SMS messages, blog or social media posts, etc., for the specific purpose of predicting a degree of complementary fit of a subject when forming a team; (2) automated identification and analysis of personality differences and creation of insight and suggested actions; (3) predictive analysis of personality to predict degree of complementary fit of a subject within a group; (4) creation of a composite, group or team-level personality profile from a collection of individual profiles; and (5) automated analysis of both individual and composite, group-level personalities to generate insights into degree of complementary fit of an individual subject with a group.

In embodiments, a service provider, such as a Solution Integrator, could offer to perform the processes described herein. In this case, the service provider can create, maintain, deploy, support, etc., the computer infrastructure that performs the process steps of the invention for one or more customers. These customers may be, for example, any business that utilizes teams. In return, the service provider can receive payment from the customer(s) under a subscription and/or fee agreement and/or the service provider can receive payment from the sale of advertising content to one or more third parties.

In still another embodiment, the invention provides a computer-implemented method for personality-based cognitive team member selection. In this case, a computer infrastructure, such as computer system 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method, comprising:
retrieving from multiple remote data resources, by a computing device, communication data authored by a participant;
processing, by the computing device, the communication data to produce formatted data;
analyzing, by the computing device, the formatted data to obtain personality trait scores for corresponding personality traits based on the formatted data;
generating, by the computing device, a user profile for the participant including the personality trait scores; and
storing, by the computing device, the user profile in a database.

2. The method of claim 1, wherein the multiple remote data resources comprise one or more selected from the group consisting of an email server, a blog server, a social media server and a database server.

3. The method of claim 1, further comprising:
obtaining, by the computing device, user profiles for respective team members, each of the user profiles including personality trait scores for the corresponding personality traits;
comparing, by the computing device, the participant's personality trait scores with the personality trait scores of each team member;
determining, by the computing device, that a difference between at least one of the personality traits of the participant and one of the corresponding personality traits of at least one respective team member meet a predetermined threshold value based on the comparing; and
recording, by the computing device, the difference in an event log as an event.

4. The method of claim 3, further comprising receiving, by the computing device, configurable parameter data, the configurable parameter data including the predetermined threshold value.

5. The method of claim 3, further comprising:
determining, by the computing device, that at least one of the plurality of user profiles of the team members has expired; and
obtaining, by the computing device, a new user profile.

6. The method of claim 1, further comprising:
obtaining, by the computing device, user profiles for respective team members, each of the user profiles including personality trait scores for corresponding personality traits;
calculating, by the computing device, mean personality trait scores for each of the plurality of personality traits across all team members to obtain a first set of team scores; and
calculating, by the computing device, mean personality trait scores for each of the plurality of personality traits across all team members plus the participant to obtain a second set of team scores.

7. The method of claim 6, further comprising:
comparing, by the computing device, the first set of team scores to the second set of team scores;
determining, by the computing device, that a differences between personality traits of the first set of team scores and corresponding personality traits of the second set of team scores meet a predetermined threshold value based on the comparing; and
recording, by the computing device, the difference in an event log as an event.

8. The method of claim 7, further comprising determining, by the computer device, that at least one personality trait score of Team B is less favorable than the corresponding personality trait score of Team A.

9. The method of claim 7, further comprising:
analyzing, by the computing device, the nature of potential conflicts indicated by the events in the event log; and
generating, by the computing device, a report based on the analysis, wherein the report includes recommendations predictive of a degree of complementary fit of the participant within a team.

10. A computer program product for personality-based cognitive team member selection, the computer program product comprising a computer readable storage medium having program instructions embodied therewith, the program instructions executable by a computing device to cause the computing device to:
obtain a user profile for a participant, the user profile including personality trait scores for corresponding personality traits;
obtain user profiles for respective team members, each of the user profiles including personality trait scores for the corresponding personality traits;
compare the participant's personality trait scores with the personality trait scores of each team member;
determine that a difference between at least one of the personality traits of the participant and one of the corresponding personality traits of at least one respective team member meets a predetermined threshold value based on the comparing; and
record the difference in an event log as an event.

11. The computer program product of claim 10, wherein the program instructions further cause the computing device to receive and store configurable parameter data, the configurable parameter data including the predetermined threshold value.

12. The computer program product of claim 10, wherein the program instructions further cause the computing device to:
determine that at least one of the plurality of user profiles of the team members has expired; and
obtain a new user profile.

13. The computer program product of claim 10, wherein the program instructions further cause the computing device to:
calculate mean personality trait scores for each of the plurality of personality traits across all team members to obtain a first set of team scores; and
calculate personality trait scores for each of the plurality of personality traits across all team members plus the participant to obtain a second set of team scores.

14. The computer program product of claim 13, wherein the program instructions further cause the computing device to:
compare the first set of team scores to the second set of team scores;
determine that a difference between personality traits of the first set of team scores and corresponding personality traits of the second set of team scores meets a predetermined threshold value based on the comparing; and
record the difference in the event log as an event.

15. The computer program product of claim 14, wherein the program instructions further cause the computing device to:
analyze the nature of potential conflicts indicated by the events in the event log; and
generate a report based on the analysis.

16. A system for personality-based cognitive team member selection, comprising:
a CPU, a computer readable memory and a computer readable storage medium associated with a computing device;
program instructions to obtain a user profile for a participant, the user profile including personality trait scores for corresponding personality traits;
program instructions to obtain user profiles for respective team members, each of the user profiles including personality trait scores for the corresponding personality traits;
program instructions to calculate mean personality trait scores for each of the plurality of personality traits across all team members to obtain a first set of team scores;
program instructions to calculate personality trait scores for each of the plurality of personality traits across all team members plus the participant to obtain a second set of team scores;
program instructions to compare the first set of team scores to the second set of team scores;
program instructions to determine that a difference between personality traits of the first set of team scores and corresponding personality traits of the second set of team scores meets a predetermined threshold value based on the comparing; and
program instructions to record the difference in an event log as an event;
wherein the program instructions are stored on the computer readable storage medium for execution by the CPU via the computer readable memory.

17. The system of claim 16, further comprising:
program instructions to compare the participant's personality trait scores with the personality trait scores of each team member;
program instructions to determine that a difference between at least one of the personality traits of the participant and one of the corresponding personality traits of at least one respective team member meets a predetermined threshold value based on the comparing; and
program instructions to record the difference in the event log as an event.

18. The system of claim 17, further comprising program instructions to receive and store configurable parameter data, the configurable parameter data including the predetermined threshold value.

19. The system of claim 17, further comprising:
program instructions to analyze the nature of potential conflicts indicated by the events in the event log; and
program instructions to generate a report based on the analysis.

20. The system of claim 19, further comprising:
program instructions to obtain communication data authored by the participant and each of the team members;
program instructions to process the communication data to produce formatted data;
program instructions to analyze the formatted data to obtain, for the participant and each of the team members, the personality trait scores for the corresponding personality traits;
program instructions to generate the user profiles of the participant and each of the team members; and
program instructions to store the user profiles in a user profile database.
